(19) United States
(12) Patent Application Publication
(10) Pub. No.: US 2017/0042955 A1
(43) Pub. Date: Feb. 16, 2017

(54) Novel Anti-Fungal Composition

(71) Applicant: Vivek Anand Parachur, Chennai, TN (IN)

(72) Inventors: Vivek Anand Parachur, Chennai (IN); Sripathy Ravichandran, Chennai (IN)

(21) Appl. No.: 15/306,421

(22) PCT Filed: Apr. 23, 2015
(86) PCT No.: PCT/IN2015/000180

§ 371 Date: Oct. 24, 2016

(30) Foreign Application Priority Data
Apr. 23, 2014 (IN) 2079/CHE/2014

(51) Int. Cl. A61K 36/185; A61K 31/122; A61K 31/352; A61K 31/7048; A61K 9/00; A61K 9/06; A61K 47/10; A61K 47/26; A61K 47/18; A61K 47/22; A61K 47/12; A61K 47/32; A61K 47/38; A61K 47/02; A61K 45/06

(52) U.S. Cl. CPC A61K 36/185; A61K 45/06; A61K 31/122; A61K 31/352; A61K 31/7048; A61K 9/0034; A61K 9/0053; A61K 9/06; A61K 47/10; A61K 47/26; A61K 47/183; A61K 47/22; A61K 47/12; A61K 47/32; A61K 47/38; A61K 47/02; A61K 2236/33

(57) ABSTRACT

The present invention discloses novel antifungal herbal composition comprising standardized ethyl acetate extracts or active ingredients isolated from ethyl acetate extract of Lawsonia inermis, for the management of fungal infections, including those caused by (Candida sp., Cryptococcosis sp., Aspergillus sp., Penicillium sp, Tinea sp and Blastomyces sp.). More particularly, the present invention discloses herbal composition comprising ethyl acetate extracts of Lawsonia inermis or the compositions comprising at least one of active ingredients selected from Lawsone & lawsoniaside and luteoline, for the management of fungal infections.

### TECHNICAL FIELD OF THE INVENTION

The present invention relates to novel antifungal herbal composition comprising standardized ethyl acetate extracts or active ingredients isolated from ethyl acetate extract of Lawsonia inermis, for the management of fungal infections, including those caused by (Candida sp., Cryptococcosis sp., Aspergillus sp., Penicillium sp, Tinea sp and Blastomyces sp.). More particularly, the present invention discloses herbal composition comprising ethyl acetate extracts of Lawsonia inermis or the compositions comprising at least one of active ingredients selected from Lawsone & lawsoniaside and luteoline, for the management of fungal infections.

### BACKGROUND AND PRIOR ART

Fungal borne infections or mycoses are most common infections among humans and animals. A variety of environmental and physiological conditions can contribute to the development of mycoses. Mycoses can be localized to skin or specific organs such as lungs, intestine or can cause serious life threatening systemic infections, which depends on various factors including immune status, virulence of infecting fungi and so on. Mycoses can be classified in to superficial, cutaneous, subcutaneous and systemic mycoses based on the site of infection. Fungal species causing infections can be structurally multicellular mold, unicellular yeast or dimorphic in nature. Opportunistic fungal infections are most difficult to treat especially with increase in drug resistance. Candidiasis, cryptococcosis and aspergillosis are frequently observed opportunistic fungal infections among immunocompromised patients.

Among the opportunistic fungal infections, Candidiasis, especially invasive Candidiasis, form the most common cause of global morbidity and mortality. Invasive Candidiasis has an attributable mortality rate of 10-49%. Candidiasis accounts for 8-10% of the total systemic nasocomial infection in United States [1-4]. Similar trend was observed in India where invasive Candidiasis dominates among the opportunistic mycosis and is the second vital cause for the co-morbidity in HIV infected patients [3]. Studies conducted at different locations in India confirm that around 40% of the HIV infected patients have Candidiasis as secondary infection. Patients with lower CD4+ cells were reported to have oral Candidiasis [66%] and pulmonary TB [60%] [5-7]. Mortality in candidemia patients in India ranges from 28-71.4% and attributable mortality rate falls between 17% and 33% [8].

Virulence of Candida sp. is attributed to many factors such as hyphal formation, surface recognition, phenotypic switching and extracellular hydrolytic enzyme production (16). Secreted aspartyl proteinases (Sap), phospholipase B enzymes, and lipases are the 3 most important extracellular hydrolytic enzymes produced by C. albicans (14, 15). Many of the pathogenic Candida species have been shown to produce active extracellular proteases, including C. albicans, C. dubliniensis, C. tropicalis and C. parapsilosis. Such proteases are responsible for digesting or distorting host cell membranes to facilitate adhesion and tissue invasion of this dimorphic opportunistic pathogen Candida (15). Another heterogeneous group of enzymes that share the ability to hydrolyze one or more ester linkage in glycerol phospholipids are called phospholipases. Although all phospholipases target phospholipids as substrates, each enzyme has the ability to cleave a specific ester bond and accordingly they are classified as A, B, C and D. Phospholipase C (PLC) hydrolyzes the phosphor-dieter bond in the phospholipids backbone to yield 1,2-diacylglycerol and, depending on the specific phospholipid species involved, phosphatidylcholine, phosphatidyl ethanolamine, etc. Inhibition of these two hydrolytic enzyme activity is confirmed to reduce the pathogenicity of Candida and can also prevent invasive candidiasis.

Antifungal treatment options have been classified into three classes, Viz, azoles, polyenes and 5-fluorocytosine. Azoles are class of compounds that inhibit the synthesis of ergosterol (the major fungal cell wall sterol component). Polyenes interact with fungal membrane sterols and 5-fluorocytosine inhibits macromolecular synthesis. Candida sp. was found to develop resistance to these antifungal agents through various drug resistance mechanisms. These mechanisms include alteration in drug target, alteration in sterol biosynthesis, reduction in the intercellular concentration of target enzyme, and over expression of the antifungal drug target.

Lawsonia inermis, also popularly known as Henna, is a small desert tree, but can also be grown as a houseplant. The henna plant is native to northern Africa, western and southern Asia; northern Australasia and tropical areas. Historically, henna was used for cosmetic purposes in Ancient Egypt or Carthage, as well as other parts of North Africa, the Horn of Africa, the Arabian Peninsula, and South Asia. The lawsone molecules in the henna leaves are responsible for the color that stains the skin.

Different types of solvent and aqueous leaf extracts of Lawsonia inermis also known as Henna were tested earlier for antifungal activity. Many of the aqueous extracts failed to exhibit antifungal activity. Solvent extracts tested included alcohols (methanol, ethanol), chloroform, benzene, ether and hexane. Among these, alcoholic extracts shows some promise, still they weren't tested further for its specific effect on Candida sp, other than inhibitory activity. (9, 10, 11, 12). One study with ethyl acetate extract of henna on Candida sp. concluded that there isn't appreciable activity of this extract and lawsone on Candida sp (13).

However, none of the prior arts discloses the extract of abundantly available Lawsonia inermis possessing significant antifungal activity with unique mode of action that can be safely used for fungal infections caused by resistant fungi in various groups of patients.

Therefore, there remains a need in the art to develop a herbal composition that is safe, cost effective and can effectively tackle fungal resistance in a human and/or animal.

Accordingly, the objective of the present invention is to provide a potential successor in the field of drugs, biopharmaceuticals and nutritional/dietary supplements, for human and/or animal application in the treatment of fungal infections including candidiasis.

### SUMMARY OF THE INVENTION

In an important aspect, the invention provides a herbal pharmaceutical composition/dietary supplement which comprises biologically effective standardized ethyl acetate extract of Lawsonia inermis, wherein the standardized ethyl acetate extract comprises total Phenolic content which includes lawsoniaside in an amount ranging from 0.00001 to 30%; total Flavonoid content which includes luteoline ranging from 0.00001 to 40%; and Lawsone content ranging from 0.00001 to 99%, together with one or more pharmaceutical excipients, for the management of fungal infections.

In a preferred aspect, the invention discloses herbal composition comprising biologically effective extract of Lawsonia species, specifically Lawsonia inermis, said biologically effective extract comprises 0.01% to 99% of at least one active compound selected from the group consisting of Lawsone & lawsoniaside and luteoline, for the effective management of fungal infections.

In yet another aspect, the invention describes ethyl acetate extract of Lawsonia inermis standardized to comprise bioactive and marker compound, Lowsone, phenolic compound-lawsoniaside and flavonoids-luteoline.

In yet another aspect, the invention provides ethyl acetate extract of Lawsonia inermis with significant antifungal activity. In one preferred aspect, the fungal infection is candidiasis.

In yet another aspect, the invention provides Lawsonia inermis extract with novel and unique mode of action against Candidia sp.

In yet another aspect, the invention provides ethyl acetate extract or fractions or compositions comprising at least one compound selected from the group consisting of Lawsone & lawsoniaside and luteoline derived from Lawsonia inermis, having antifungal mode of action against di-morphic fungi being through germ tube inhibitory activity of Lawsonia inermis extract.

In a preferred aspect, the invention provides herbal compositions comprising extracts or fractions or at least one compound selected from the group consisting of Lawsone & lawsoniaside and luteoline having anticandidal properties and mechanism of action being the inhibition of key virulent enzymes—proteases (SAP3) and phospholipase C of Candida sp.

In yet another aspect, the invention provides compositions comprising Lawsone; lawsoniaside and luteoline having anticandidal properties with the mechanism of action being the inhibition of key constitutive enzymes—aspartate semi-aldehyde dehydrogenase, N myristoyltrasnferases and lumazine synthase of Candida sp.

In yet another aspect, the invention provides herbal composition comprising Lawsoniainermis extract formulated in liquid, semisolid or solid dosage form. The invention also provides the Lawsonia inermis extract for preparation of medicament useful for oral, topical, ocular, vaginal, otic and dermal application.

In one preferred aspect, the invention provides semisolid dosage forms such as oral, dermal, topical, ocular, otic and vaginal gels. In another preferred aspect, the invention provides liquid and solid dosage forms for oral, topical, ocular, vaginal, otic and dermal application.

The anti-candidal composition according to the invention can also be used in feminine hygiene products, including maxi pads, ultra-thin pads, and feminine wipes. Further, the anti-candidial composition according to the invention can also be prepared as a suppository; tooth paste; mouth wash; as an adjunct to female and male contraceptive devices; as an adjunct to vaginal contraceptives and lubricants.

In yet another embodiment, the invention encompasses pharmaceutical compositions comprising lawsone Lawsonisides and Luteoline either alone or in combination in an amount of 0.0001% to 99.99% in association with one or more pharmaceutical excipients for the management of fungal infections.

The dosage forms of the invention may be formulated by the methods known in the art using pharmaceutical carriers and excipients such as diluents, polymers, surfactants, solubilizers, colors, flavors, pH adjusting agents, surfactants and the like. The dosage forms may be formulated using aqueous, non-aqueous and alcohols.

In yet another aspect, the invention provides herbal compositions comprising Lawsonia inermis extract, for various therapeutic, preventative and general health supplement applications in animals and human beings.

In a further another aspect, the invention provides a Lawsonia inermis extract for use as pharmaceutical, dietary supplements, nutraceuticals/health supplements/general supplements or OTC products.

In yet another aspect, the invention provides use of extract containing lawsone and other signature compounds) in combination with known drugs such as azoles (fluconazole, ketaconazole, clotrimazole, corbimazole, ravuconazole and posacconazole); Amphotericins and/or echinocandins (Echinocandin, caspofungin, micafungin, anidulafungin and cilofungin) for the management of local and systemic fungal infections.

### DESCRIPTION OF DRAWINGS

FIG. 1: Anticandidal activity of Lawsone against Candida albicans.

FIG. 2: Germ tube inhibitory activity of Henna ethyl acetate extract on Candida albicans.

FIG. 3: TLC analysis of Henna extract and standard Lawsone (Dimension of plate 4X9, Rf=0.71)

FIG. 4: HPLC analysis of standard Lawsone (Rt: 8.68 mins)

FIG. 5: HPLC analysis of Henna extract with Lawsone peak (Rt: 8.41 mins)

FIG. 6: Effect of Henna extract and Lawsone on the Protease enzyme activity of C. albicans.

FIG. 7: Native gel-PAGE profile of aspartate dehydrogenase activity of Lawsonia inermis treated C. albicans in comparison with control and DMSO control treatment.

### DETAILED DESCRIPTION OF THE INVENTION

The invention will now be described in detail in connection with certain preferred and optional embodiments, so that various aspects thereof may be more fully understood and appreciated.

Accordingly in a preferred embodiment, the invention describes antifungal composition, which effectively combats fungal infections in human or an animal. In one preferred embodiment, the fungal infection is candidiasis.

Thus the present invention is directed towards development of biologically effective extract of Lawsonia species specifically Lawsonia inermis (Henna) extract, which possesses antifungal activity. According to the present invention, ethyl acetate extract of Lawsonia inermis was found to have effective anti-candidal activity. Extract has been profiled and identified to have marker compounds including pigments (lawsone), tannins and other flavonoids and purified Lawsone has been further investigated as one of the active compound responsible for the anti-candidal activity and confirmed to possess significant anti-candidal activity.

Accordingly, in an embodiment, the present invention provides the extract of Lawsonia inermis with unique mechanism of action which include germ tube inhibitory activity and inhibition of virulent (proteases and phospholipases) and constitutive (aspartate dehydrogenases, lumazine synthase and N Myristoyil transferases) enzymes. This mechanism of action is different from other marketed anti-fungal agents such as azoles, which inhibit the synthesis of ergosterol (the main fungal sterol); polyenes, which interact with fungal membrane sterols physicochemically; and 5-fluorocytosine, which inhibits macromolecular synthesis.

According to preferred embodiment, the invention provides a herbal pharmaceutical composition/dietary supplement which comprises biologically effective standardized ethyl acetate extract of Lawsonia inermis, wherein the standardized ethyl acetate extract comprises total Phenolic content which includes lawsoniaside in an amount ranging from 0.00001 to 30%; total Flavonoid content which includes luteoline ranging from 0.00001 to 40%; and Lawsone content ranging from 0.00001 to 99%, together with one or more pharmaceutical excipients, for the management of fungal infections.

According to one preferred embodiment, the invention provides a herbal composition comprising biologically effective extract of Lawsonia inermis, said biologically effective extract comprises 0.01% to 99% of at least one compound selected from the group consisting of Lawsone & lawsoniaside and luteoline together with one or more pharmaceutical excipients, for the management of fungal infections. The biologically effective extract is ethyl acetate extract of Lawsonia inermis.

In yet another embodiment, the invention provides herbal composition comprising biologically effective extract of Lawsonia inermis standardized to comprise at least one of Lawsone & lawsoniaside and luteoline along with tannins

In a further embodiment, the invention provides a composition comprising Lawsone together with one or more pharmaceutical excipients.

In yet another embodiment, the invention provides herbal composition comprising ethyl acetate extract of Lawsonia inermis containing bioactive compounds naphthoquinones (Lawsone, lawsoniaside), tannis and flavonoids (luteoline) for use in the treatment of systemic and localized fungal infections, more specifically, oral and vaginal candidiasis.

In yet another embodiment, the invention describes an Lawsonia inermis extract with novel and unique mode of action against candidiasis, which include germ tube inhibitory activity and inhibition of virulent (proteases and phospholipases) and constitutive (aspartate dehydrogenases, lumazine synthase and N Myristoyiltransferases) enzymes.

In an alternate embodiment, the invention provides use of extract containing lawsone and other signature compounds) in combination with known drugs such as azoles (fluconazole, ketaconazole, clotrimazole, corbimazole, ravuconazole and posacconazole); Amphotericins and/or echinocandins (Echinocandin, caspofungin, micafungin, anidulafungin and cilofungin) for the management of local and systemic fungal infections.

In yet another embodiment, the invention provides herbal composition comprising Lawsonia inermis extract formulated in the form of liquid, semisolid or solid dosage forms using one or more pharmaceutical excipients/carriers/diluents. The pharmaceutical excipients/carriers/diluents are selected from the group consisting of polymers, diluents, binders, fillers, disintegrants, lubricants, colors, flavors and the like.

The invention also provides the Lawsonia inermis extract for preparation of medicament for use in oral, topical, ocular, vaginal, otic, dermal application. The pharmaceutical carriers/excipients used for the preparation of medicaments may be selected from the conventional excipients known to a person skilled in the art. The methods of formulating the composition suitable for oral, topical, ocular, vaginal, otic, dermal application may be selected from the conventional methods known to a person skilled in the art.

The anti-candidal composition according to the invention can also be used in feminine hygiene products, including maxi pads, ultra-thin pads, and feminine wipes. Further, the anti-candidial composition according to the invention can also be prepared as a suppository; tooth paste; mouth wash; as an adjunct to female and male contraceptive devices; as an adjunct to vaginal contraceptives and lubricants.

In yet another embodiment, the invention encompasses pharmaceutical compositions comprising lawsone, Lawsonisides and Luteoline either alone or in combination in an amount of 0.0001% to 99.99% in association with one or more pharmaceutical excipients, for the management of fungal infections.

In yet another embodiment, the invention provides Lawsonia inermis extract for the preparation of various therapeutic, preventative and general health supplements in animals and human beings.

In yet another embodiment, the invention describes a Lawsonia inermis extract for use as Pharmaceutical, dietary supplements or nutraceuticals/health supplements/general supplements/OTC products.

Therefore, the invention includes within its scope, the compositions and the use of compounds in the biologically active extract of Lawsonia inermis, including compounds, Lawsone & lawsoniaside and luteoline having anti-fungal and anti-candidal activity; the synthetic forms of such compounds and, such compounds prepared by appropriate synthesis.

In yet another embodiment, the invention provides process for preparation of ethyl acetate extract of Lawsonia inermis. According to this process the dried green leaves are converted preferably into a powder by crushing or grinding prior to being treated with ethyl acetate solvent to extract there from the active ingredients. Preferably, powder of fine particles is used and the powder is macerated with the ethyl acetate solvent to obtain a dark green colored solution which has the desired concentration of dissolved biologically active extract. Accordingly, the process for preparation of biologically effective standardised ethyl acetate extract of Lawsonia inermis, which comprises;
a) soaking the crushed powder of green leaves of Lawsonia inermis with ethylacetate in a ratio of 10:1 to 1:10 overnight in air tight container followed by pulverization;
b) extracting the pulverised crushed leaves with aqueous solvent at a temperature below 60° C. for a period of time sufficient to produce a dark green colored solution which has the desired concentration of dissolved biologically active extract; and
c) recovering the biologically active extract after evaporating the solvent.

In yet another embodiment, the invention provides evaluation of biological activity of the extract of Lawsonia inermis against various fungal species such as C. albicans and C. glabrata and the zone of inhibition at various concentrations. In yet another embodiment, the Anticandidal Activity of Lawsone is evaluated and the zone of inhibition at various concentrations. According to the above studies, the Lawsone in ethylacetate extract of Lawsonia inermis is responsible for the Anticandidal activity. (cf. table 1 and 2). Thus the invention further provides compositions comprising Lawsone together with one or more pharmaceutical excipients.

The following examples, which include preferred embodiments, will serve to illustrate the practice of this invention, it being understood that the particulars shown are by way of example and for purpose of illustrative discussion of preferred embodiments of the invention.

### Example 1

### Extract Preparation

A source of the biologically active extract of the present invention comprises Lawsonia inermis plant leaf extract.

A preferred source of the biologically active material for use in the composition of the present invention is a part of the Indian Lawsonia inermis plant tree.

The preferred Lawsonia inermis tree parts for use in the present invention is shade dried green leaves. The dried green leaves are converted preferably into a powder by crushing or grinding prior to being treated with ethyl acetate solvent to extract there from the active ingredients. Preferably, a powder of fine particles is used and the powder is Mac®rated with the ethyl acetate solvent. Any suitable method involving the use of the ethyl acetate solvent of the present invention can be used to extract the biologically active ingredients from the green leaves of the aforementioned Lawsonia inermis.
Ratio of Ethyl acetate to dry henna leaves for extract preparation shall be in the range of 1:10 to 10:1, while the most optimum ratio shall be 1:3. The ethyl acetate solvent for use in the extraction is used in the ratio of 1:3 of the powdered dry leaves. The powdered leaves are soaked in the solvent for overnight in air tight container and then pulverized to fine powder using suitable external aids (mortar and pestle). The extraction should be conducted under temperature conditions which reduce the tendency of the biologically active ingredients comprising the extract to decompose. It is preferable that the temperature of the aqueous solvent be no greater than about 60° C. and preferably in the range of about 4° C. to about 20° C. When the solvent has a temperature other than ambient or room temperature (about 23° C.), refrigerating or heating equipment can be used to lower or raise the temperature of the solvent to the desired operating temperature. The extracting conditions which involve the ethyl acetate solvent at the desired temperature should be carried out for a period of time sufficient to produce a dark green colored solution which has the desired concentration of dissolved biologically active extract; the extract in a dark brownish green paste form can be recovered from the solution by evaporating the liquid solvent. Prior to evaporation, the solution can be filtered, as may be needed, to remove therefrom solid impurities.

The ethyl acetate extract is characterised for the presence of phytoconstituents, tannins, Lawsone & lawsoniaside and luteoline and its concentrations. The total phenolic content which includes lawsoniaside; total flavonoid content which includes luteoline and lawsone content are shown in table 7. According to this phytochemical analysis, the standardised ethyl acetate extract comprises total Phenolic content in an amount ranging from 0.00001 to 30%; total Havonoid content ranging from 0.00001 to 40%; and Lawsone content ranging from 0.00001 to 99%. In addition to this presence of tannins, coumarines and quinines have also been confirmed.

The invention includes within its scope the use of compounds comprising the biologically active extract, including compounds comprising the anti-candidal fraction of the extract, and synthetic forms of such compounds, that is, such compounds prepared by appropriate synthesis.

### Example 2

### Evaluation of Anti-Candidal Activity of Lawsonia inermis Extract

Following in vitro study showed effective dose dependent anti-candidal activity for extract as well as for Lawsone treatment.

### Antifungal Activity by Modified Kerby-Bauer Method (Bauer et al., 1966)

Susceptibility disks (5 mm in diameter) were impregnated with 0.5 mg of desired compound(s)/extract dissolved in DMSO, and placed on Sabouraud dextrose agar plates separately inoculated with the C. albicans and C. glabrata. After 48 hrs of incubation at 37° C., plates were observed for zone of inhibition. Disc containing only DMSO was used as a negative control.
   Anticandidal activity against Candida species:
Dose dependent anti-candidal activity was observed up on treatment using Lawsoniainermisextract against tested C. albicans and C. glabrata (Table 1).

TABLE 1
Anti-candidal activity of extract against C. albicans and C. glabrata.

| | |
|---|---|
| Concentration of | Zone of Inhibition (mm) |

| Sl. No. | Extract | | C. albicans | C. glabrata |
|---|---|---|---|---|
| 1. | 0 | mg (Control) | NIL | NIL |
| 2. | 0.25 | mg | 9 | 7 |
| 3. | 0.5 | mg | 13 | 9 |
| 4. | 0.75 | mg | 15 | 11 |
| 5. | 1 | mg | 17 | 14 |

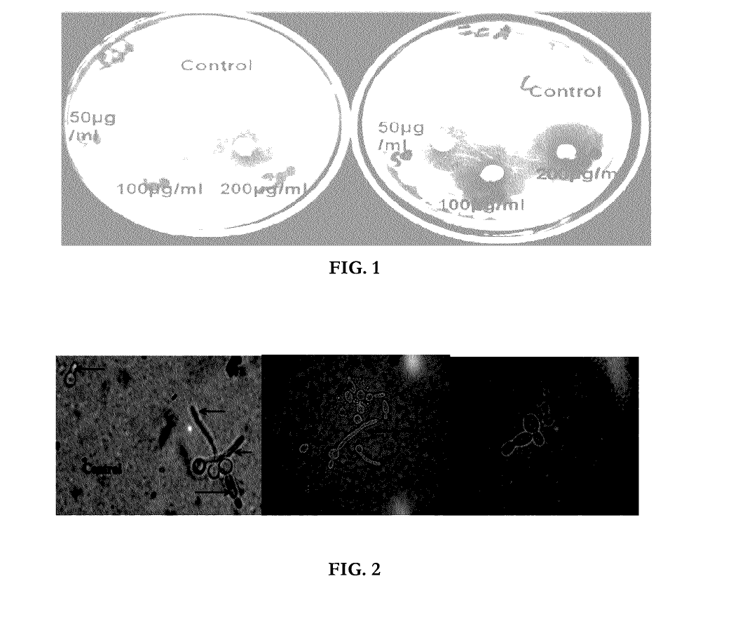

Anticandidal Activity of Lawsone:
Complete inhibition of candidal growth was found in C. albicans upon lawsone treatment in all three concentrations (50, 100 and 200 μg/ml) tested as shown in FIG. 1 and table 2.

TABLE 2
Anti-candidal activity of Lawsone against C. albicans.

| | Concentration of | Zone of Inhibition (mm) |
|---|---|---|
| Sl. No. | Extract | C. albicans |
| 1. | 50 μg | 14 |
| 2. | 100 μg | 16 |
| 3. | 200 μg | 17 |

Germ tube inhibitory activity:
   Dried plant extract was taken at 500 μg concentration in a test tube and resuspended in 10 μl of DMSO. 990 μl of bovine serum was added and loop full of mid log phase (105 to 107 cells/ml) Candida culture was inoculated in the serum and incubated for three hours at 37° C. Growth of germ tube was observed under the microscope (Krestchmar et al¹⁷., 1999)(FIG. 2).
   Germ tube formation is a key aspect for virulence in dimorphic fungi. Germ tube inhibitory activity in Candida albicans was found upon Henna ethyl acetate extract treatment. Hence this extract can be used to control invasive candidiasis.

### In Silico

Docking studies against following Candida sp. targets using Lawsone, lawsoniaside and luteoline
   Proteases
   Phospholipases
   Lumazine synthase
   N Myristoyltransferase&
   Aspartate semi-aldehyde dehydrogenase
Among the compounds screened, lupeoline, lawsone, aesculetin and lowsoniaside, were found to have good affinity towards screened Candida targets (Table 3). Lupeoline has multi-target potency with binding affinity towards 4 out of 6 Candida targets screened. Aesculetin and lawsone showed binding affinity towards 3 Candida targets. Lawsoniaside is the only compound to have affinity to 2H6T among the compounds screened. In addition to this Lawsoniaside has good affinity to 2JFB. Based on the docking score and docking energy it can be inferred that lawsone and lupeoline has moderate Candida myristoyltransferase, lumazine synthase and phospholipase inhibitory activity. Very low glide energy and docking score indicates that solamargine and lawsoniaside has very good inhibitory activity against asparatate semi aldehydede hydrogense and lumazine synthase. Lawsoniaside is the only enzyme to have very good binding energy and docking score to Candida enzyme SAP3.

TABLE 3
Insilico Extra Precision (GLIDE-Schrodinger) Docking Study Results of Ligands
from Henna plant leaves against Candida sp. Targets.

| | | Phytochemical | Plant | Docking | |
|---|---|---|---|---|---|
| Sl. No. | Target (PDB ID) | ligand | Source | score | Glide energy |
| 1. | N- | lupeoline | L. inermis | −3.342687 | −31.735758 |
| | Myristoyltransferase | lawsone | L. inermis | −2.603266 | −24.143367 |
| | (1NMT) | aesculetin | L. inermis | −2.408067 | −23.024805 |
| 2. | Aspartate | lupeoline | L. inermis | −5.860507 | −37.422997 |
| | semialdehyde | aesculetin | L. inermis | −5.246416 | −24.084277 |
| | dehydrogenase | | | | |
| | (3HSK) | | | | |
| 3. | Secreted | Lowsoniaside | L. inermis | −10.120195 | −58.117896 |
| | AsparticProteinase | | | | |
| | 3 (2H6T) | | | | |
| 4. | Lumazine Synthase | lowsoniaside | L. inermis | −6.48773 | −46.796851 |
| | (2JFB) | lawsone | L. inermis | −3.385353 | −34.573629 |
| | | lupeoline | L. inermis | −2.885515 | −32.834679 |
| 5. | Phospholipase C | lupeoline | L. inermis | −4.743477 | −33.189219 |
| | | lawsone | L. inermis | −4.147062 | −24.900805 |
| | | Aesculetin | L. inermis | −3.257346 | −26.639346 |

### In Silico Induced Fit Docking Studies—Candida Targets:

Induced fit docking study inferred that lawsone can be a very good inhibitor of all three Candida targets (myristyltransferase, phospholipases and lumazine synthase) as indicated by the GLIDE energy and GLIDE score. Lawsone individually formed 4 hydrogen bonds with all three Candida targets. The site of interaction and bond distance for hydrogen bonding with respective targets were described in table 4. With less GLIDE energy and more number of hydrogen bonds lawsone shall be considered to be an effective inhibitor of all three Candida targets.

TABLE 4
Insilico Induced Fit (GLIDE-Schrodinger) Docking Study Results of Lawsone
from Henna plant leaves against Candida sp. Targets.

| | | | | | GLIDE |
|---|---|---|---|---|---|
| | | | H-Bond | Docking | Energy |
| Sl. No | Target | Interactions | (Å) | Score | (KCAL\Mol) |
| 1 | N- | TYR 335 (OH . . . O) | 2.81 | −7.18 | −38.87 |
| | myristyltransferase | (OH . . . O) LEU 451 | 2.85 | | |
| | (1NMT) | TYR 119 (OH . . . O) | 2.74 | | |
| | | TYR 107 (OH . . . O) | 2.89 | | |
| 2 | Phospholipase | (OH . . . N) HIS 76 | 2.96 | −7.47 | −33.35 |
| | | GLN 39 (NH . . . O) | 3.18 | | |
| | | (OH . . . O) SER 37 | 2.84 | | |
| | | (OH . . . O) ASN 243 | 2.84 | | |
| 3 | Lumazinesynthase | (OH ... O) VAL 90 | 2.96 | −7.00 | −32.58 |
| | (2JFP) | (OH . . . O) TRP 27 | 3.33 | | |
| | | SER 61 (OH . . . O) | 2.91 | | |
| | | SER 61 (NH . . . O) | 2.89 | | |

### Phytochemical Analysis of Extract:

Extract has been characterized to identify active compound(s) using chromatographic techniques such as TLC (Thin layer chromatography) and HPLC (High Performance liquid Chromatography).

### TLC:

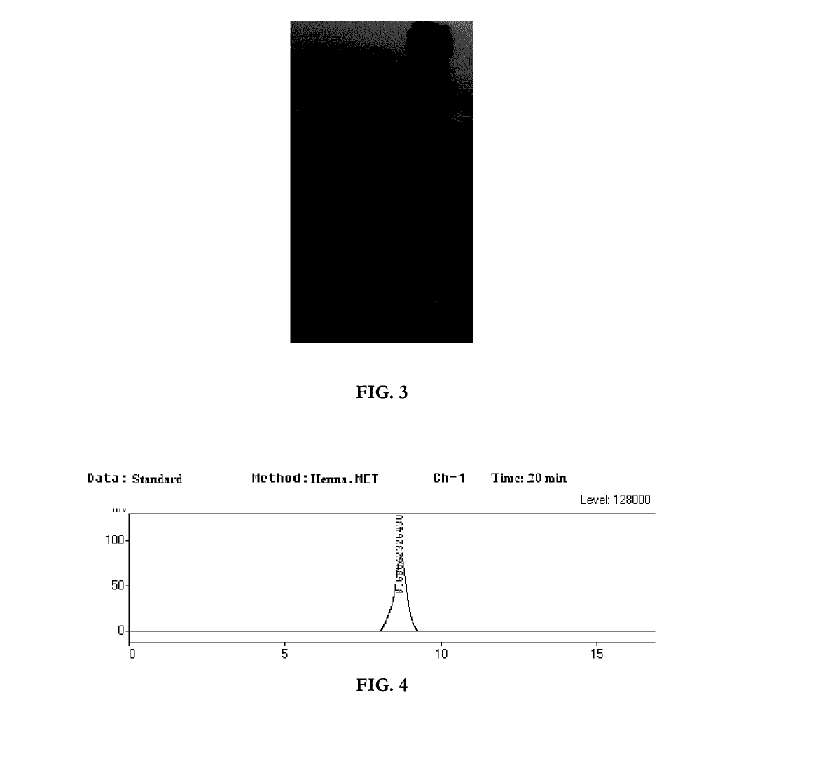

TLC has been performed using different combinations and concentrations of extract to check the presence of Lawsone, one of major bioactive compound identified to have enzyme inhibitory activity, as described earlier in the in silico docking studies. Different combinations and proportions of mobile phase has been tried to separate and identify the presence of Lawsone. Different solvents used for optimizing mobile phase includes, chloroform, ethyl acetate, acetic acid, toluene, ethanol, petroleum ether, hexane and benzene. Mobile phase has been optimized to Toluene-Ethyl acetate-Acetic acid (5:4:1). Lawsone sigma standard has been used as marker to track the presence of active lawsone in the Henna extract. As described in FIG. 3, an yellowish-orange colored band with similar Rf as that of standard Lawsone was found to be present in the TLC plate. This indicates the presence of Lawsone in the tested ethyl acetate extract of henna leaves.

### HPLC Analysis of Henna Extract

HPLC analysis of extract and standard lawsone was performed under the HPLC conditions described below:

### Column: Luna 5u RP-C18 (Phenomenex)

Mobile Phase: Methanol-Water acidified with 0.06% of trifluro acetic acid (48:52, v/v)
Flow rate: 1 ml/min

### Wavelength: 248 nm

20 μl of extract and standard was injected into a Luna 5u RP-C18 (250×4.6 mm) HPLC column containing 5 μm wide pore (100 Å) beads and was fractionated with a mixture of Methanol-Water (48:52, v/v) acidified with 0.06% trifluoro acetic acid as mobile phase. The separation was performed using isocratic elution (0-20 mins) with a flow rate of 1 ml mM-1. The elution was monitored using a Shimadzu Diode-Array Detector SPD-10A VP operating at a fixed wavelength 248 nm. The lawsone standard was purchased from Sigma chemical Co., USA.

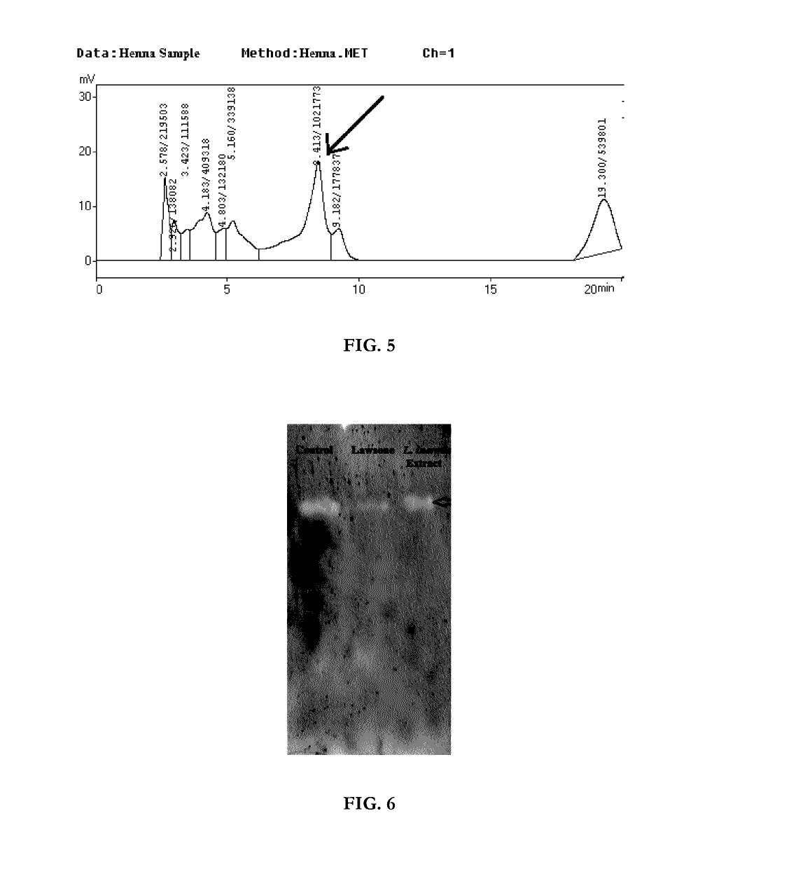

Standard lawsone peak was found at Rt 8.6 mins (FIG. 4). Extract showed several peaks and an identical peak similar to Lawsone standard was also found at similar Rt (8.41 mins) (FIG. 5). This indicates the presence of lawsone in the extract. The concentration of Lawsone in the extract was found to be 4 to 7 μg/ml of extract.

Following in vitro study details are conducted on the extract and Lawsome
Protease inhibitory activity study
   Stationary phase culture of C. albicans was used for SAP inhibition assay. SD broth consisting 100 μl of C. albicans, different concentration of extracts ranging from 0.5 mg/ml-2 mg/ml and the total volume is made up into one ml by SD broth. The test tubes were incubated at 37° C. for 6 hours with 140 rpm. After exposure of compounds the tubes were centrifuged at 5000 rpm for 5 mm. The supernatant was discarded and the obtained pellet was washed thrice with PBS (pH 7.2). Pellet was again mixed with 100 μl of SD broth and placed in SAP induction medium (23.4% Yeast Carbon Base, 2% Yeast extract and 4% BSA, pH 5.0) containing sterile filter paper discs. The plates were incubated at 37° C. for 3-5 days for proteolysis, after incubation the plates were flooded with 20% TCA (10 min), stained with 1.25% amido black dissolved in MeOH containing 10% acetic acid (10 min) and destained with 15% acetic acid (10 mm) and observed clear zone around the colonies. Pz (zone of proteolysis) ratio was measured between the control and compound treated colonies (GirishKumar et al., 2006)¹⁸.
   C. albicans protease enzyme inhibitory potential of Lawsoniainermis extract was tested at two different concentrations 500 μg/ml and 750 μg/ml. Extract treatment resulted in dose depended inhibition of protease enzyme. When compared to control, extract treatment resulted in 27% and 33% protease inhibition, at tested concentrations 500 μg/ml and 750 μg/ml respectively (Table 5).

TABLE 5
Protease inhibitory activity of Lawsonia inermis extracts
against C. albicans

Zone of Inhibition

| | | 500 μg/ml | 750 μg/ml |
|---|---|---|---|
| Sl. No | Organism | (% Inhibition) | (% Inhibition) |
| 1. | C. albicans | 6.0 mm (27) | 6.5 mm (33) |

Protease inhibitory activity—native gel electrophoresis:
Detection of proteinase inhibitory activity of extract and Lawsone in C. albicans was determined using Zymogram method described by Schmidt et al (1988) with minor modification. C. albicans NCIM 3074 culture was grown in Sabouraud broth at 37° C. for 12 h. The culture was centrifuged at 7000 rpm for 5 mm to pellet the cells. The cell pellet was washed twice with normal saline and centrifuged to collect the pellets. 100 μL of Candida albicans cell suspension was added to proteinase induction medium containing 1% glucose, 3% yeast carbon base and 1% BSA (Bovine Serum Albumin). 500 μg mL-1 ethyl acetate extract of henna leaves and 100 μg mL-1 Lawsone separately dissolved in dimethyl sulfoxide. This was added to respective test tubes immediately after the inoculation of culture. The mixture was incubated for 24-48 h at 37° C. in a shaker at 150 rpm. Culture broth was centrifuged at 7000 rpm for 15 min to remove cells. Cells were lysed and cell lysis was used for zymographic studies after protein estimation. Zymogram was performed with 1% casein incorporated in the acrylamide gel to detect the proteinase inhibition activity. The proteinase inhibitory activity was indicated by no cleavage of casein (no band formation) and it can be compared to complete cleavage of casein by proteinases (white band formation) in control groups. As in FIG. 6, Zymogram results show complete degradation of casein (thick white band formation) in control samples and intact Casein (no band/less intense band formation) in treated samples (Extract and Lawsone treatment). This indicates protease inhibitory activity of activity of extract and Lawsone.
Phospholipase inhibitory activity—Spectrophotometric Analysis:
Phospholipase activity of C. albicans of control and drug treated experiments was carried out using the protocols of Leidich et al. Briefly assay mixture consists of 1 ml of sample, 1 ml of 60% perchloric acid and heated in a sand bath at 200 C until the solution turns colourless. Subsequently, 1 ml of 60% perchloric acid was added and the final volume was made to 9.1 ml using distilled water to this 0.5 ml of molybdate II reagent and 0.4 ml ANSA reagent mixture was added. The tubes were shaken well and heated in a boiling water bath for 8 mm exactly. The blue color developed was read using spectrophotometer at 680 nm.

TABLE 6
Protease inhibitory activity of Lawsonia inermis extracts against
C. albicans

% Inhibition of Phospholipase
Enzyme Activity (O.D)

| | Henna Extract | Lawsone |
|---|---|---|
| Organism | (500 μg/ml) | (100 μg/ml) |
| C. albicans | 44.45% (0.05) | 77.78% (0.02) |

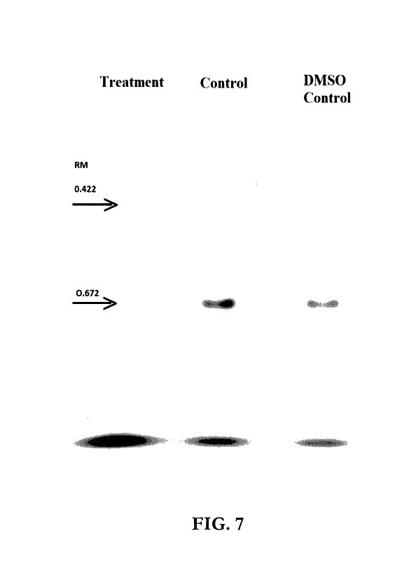

Spectroscopic analysis of change in phospholipase activity of C. albicans after treatment with lawsone and extract respectively has been tabulated in table 6. Both henna extract and lawsone showed phospholipase inhibitory activity. Percentage inhibition of phospholipase activity when compared to Control was found to be 44.5% for henna extract (500 μg/ml treatment) and 77.78% for Lawsone (100 μg/ml treatment).
Aspartate semi-aldehyde dehydrogenase inhibitory activity study
   Lawsoniainermis extract was found to completely inhibit aspartate semialehyde dehydrogenase (ASADH) activity in C. albicans. Untreated control and DMSO control C. albicans group showed two distinct bands at Rm 0.422 and 0.672, with a later prominent band. Surprisingly Lawsoniainermis (500 μg/ml) treatment completely inhibited ASADH expression in C. albicans(FIG. 7).

### Example 3

TABLE 7
Phytochemical composition of ethyl acetate extract of Lawsonia Inermis

| | Extract | | Result |
|---|---|---|---|
| Sl. No. | Components/ml | Assay method | Concentration |
| 1. | Total Phenolic content | Folin | 16.55 μg/ml |
| | which includes | Ciocalteu | |
| | lawsoniaside | method | |
| 2. | Total Flavonoid | Aluminium | 392 μg/ml |
| | content which includes | chloride | |
| | luteoline | colorimetric | |
| | | assay | |
| 3. | Lawsone | HPLC analysis | 24.32 mg/ml |

In addition to this presence of tannins, coumarines and quinines have been confirmed.

### Example 4

### Anticandida Oral and Vaginal Gel Composition

(a) Oral Gel Composition

| Sl. | | Weight |
|---|---|---|
| No. | Ingredients | (gms) |
| 1 | Henna Extract/Lawsone | 2.50 |
| 2 | PEG-400 | 2.43 |
| 3 | Tween 80 | 26.48 |
| 4 | Disodium EDTA | 0.05 |
| 5 | Sodium Saccharin | 0.50 |
| 6 | Mixed fruit flavour | 0.50 |
| 7 | Sodium methyl paraben | 0.50 |
| 8 | HPMC 4000 cps | 0.50 |
| 9 | Carbopol 974P | 2.09 |
| 10 | Sodium hydroxide | 0.25 |
| 11 | Water | 64.21 |
| | Total | 100.00 |

Final Product pH is 6-7

### Procedure—Manufacturing (Oral Gel)

Step 1:
The required quantities of all the ingredients like disodium EDTA, Sodium saccharin, Mixed fruit flavour, Sodium methyl paraben, HPMC 905H-100000, Carbopol 974P, Sodium hydroxide, Purified water are weighed accurately.

Step 2:
1. Collected required quantity of fresh purified water, in a container & boiled it. Added accurately weighed quantities of the Disodium EDTA, Sodium Saccharin, Sodium methyl paraben & Sodium hydroxide to the boiled water in container followed by stirring until completely dissolved (Solution 2).
2. Transferred the solution 2 from the container in to Planetory mixer.
3. Added weighed quantity of HPMC followed by carbopol 974P in to planetary mixer slowly and at constant speed while mixing at optimal rpm until gel base is formed.
4. Added weighed quantities of Flavour in to the mixing vessel of planetary mix containing gel base.
5. Adjusted the pH between 6 and 7.

Step 3:
1. Weighed the required quantity of gel base in planetory mixer.
2. Weighed the required quantity of Actives (henna extract/lawsone) dissolved in surfactant(s).
3. Mixed the Actives dissolved in surfactants with the gel base until uniformly dispersed fine gel was formed.

(b) Vaginal Gel Composition

| Sl. | | |
|---|---|---|
| No. | Ingredients | Weight (gms) |
| 1 | Henna Extract or Lawsone | 2.50 |
| 2 | PEG-400 | 2.43 |
| 3 | Tween 80 | 26.48 |
| 4 | Disodium EDTA | 0.05 |
| 5 | Sodium methyl paraben | 0.50 |
| 6 | HPMC 4000 cps | 0.50 |
| 7 | Carbopol 974P | 2.09 |
| 8 | Sodium hydroxide | 0.10 |
| 9 | Water | 65.35 |
| | Total | 100.00 |

Final Product pH is pH 4-6

### Procedure—Manufacturing (Vaginal Gel)

Step 1:
Weighed accurately the required quantities of all the ingredients like disodium EDTA, Sodium methyl paraben, HPMC 905H-100000, Carbopol 974P, Sodium hydroxide, Purified water.

Step 2:
1. Collected fresh purified water of required quantity in a container & boiled it. Added accurately the weighed quantities of Disodium EDTA, Sodium methyl paraben & Sodium hydroxide to the boiled water in container followed by stirring until to achieve completely dissolved Solution.
2. Transferred the solution from the container in to Planetary mixer.
3. Added weighed quantity of HPMC followed by carbopol 974P in to planetary mixer slowly and at constant speed while mixing at optimal rpm until gel base is formed.
4. Adjusted the pH of gel base between 4 and 6

Step 3:
5. Weighed the required quantity of gel base in planetary mixer.
6. Weighed the required quantity of Actives (henna extract/lawsone) dissolved in surfactant(s).
7. Mixed the Actives dissolved in surfactants to the gel base until uniformly dispersed fine gel was formed.

### REFERENCE

1. Bassetti M., E. Righi, A. Costa, R. Fasce, M. P. Molinari, R. Rosso, F. B. Pallavicini, and C. Viscoli. 2006. Epidemiological trends in nosocomial candidemia in intensive care. BMC Infect. Dis. 6:21-26.
2. Pfaller M. A., and D. J. Diekema. 2007. Epidemiology of invasive candidiasis: a persistent public health problem. Clin. Mirobiol. Rev. 20:133-163.
3. Pfaller M. A., P. G. Pappas, and J. R. Wingard. 2006. Invasive fungal pathogens: Current epidemiological trends. Clin. Infect. Dis. 43:S3-14.
4. Richardson M. D. 2005. Changing patterns and trends in systemic fungal infections. J. Antimicrob. Chemother. 56: [Suppl. S1.]i5-i11.
5. Kumar C. P. G., T. Menon, S. Rajasekaran, B. Sekar, and D. Prabu. 2008. Carriage of Candida species in oral cavities of HIV infected patients in South India. Mycoses. 52:44-48.
6. Solomon S. S., C. S. Hawcroft, P. Narasimhan, R. Subbaraman, A. K. Srikrishnan, A. J. Cecelia, M. S. Kumar, S. Solomon, J. E. Gallant, and D. D. Celentano. 2008. Comorbidities among HIV-infected injection drug users in Chennai, India. Indian J. Med. Res. 127:447-452.
7. Wadhwa A., R. Kaur, S. K Agarwal, S. Jain, and P. Bhalla. 2007. AIDS-related opportunistic mycoses seen in a tertiary care hospital in North India. J. Med. Microbiol. 56:1101-1106.
8. Chakrabarti A., S. S. Chatterjee, and M. R. Shivaprakash. 2008. Overview of opportunistic fungal infections in India. Jpn. J. Med. Mycol. 49:165-172.
9. Habbal O. A., A. A. Al-Jabri, H. Abdulghaffar, El-Hag, Z. H. Al-Mahrooqi and N. A. Al-Hashmi. 2005. In vitro antimicrobial activity of Lawsoniainermis Linn (henna): A pilot study on the Omani henna. Saudi Med J. 26. p. 69-72.
10. Dinesh Babu P. and R. S. Subhasree. 2009. Antimicrobial Activities of Lawsoniainermis—A Review. Academic Journal of Plant Sciences. Vol. 2.231-232.
11. Rahmoun N. M., Z. Boucherit-Atmani, M. Benabdallah, K. Boucherit, D. Villemin, N. Choukchou-Braham. 2013. Antimicrobial Activities of the Henna Extract and Some Synthetic Naphthoquinones Derivatives. American Journal of Medical and Biological Research. 1. p. 16-22.
12. Habbal, Al-Jabri and El-Hag. 2007. Antimicrobial properties of Lawsoniainermis (henna): a review. Australian Journal of Medical Herbalism. 19.
13. Rahmoun M. N., M. Benabdallah. D. Villemin. K. Boucherit., B. Mostefa-Kara., C. Ziani-Cherif and N. Choukchou-Braham. 2010. Antimicrobial screening of the Algerian Lawsoniainermis (henna). Der PharmaChemica. 2. p. 320-326.
14. Ghannoum M. A., 2000. Potential Role of Phospholipases in Virulence and Fungal Pathogenesis. Clinical microbiology reviews. 13. p. 122-143.
15. Naglik J. R., S. J. Challacombe and B. Hube. 2003. Candida albicans secretedaspartylproteinases in virulence and pathogenesis. Microbiology and molecularbiology reviews. 67. p. 122-143.
16. Calderone, R. A., and W. A. Fonzi. 2001. Virulence factors of Candida albicans. Trends Microbiol. 9. p. 327-335.
17. Kretschmar M, B. Hube, T. Bertsch, D. Sanglard, R. Merker, M. Schroder, H. Hof and T. Nichterlein. 1999. Germ tubes and proteinase activity contribute to virulence of Candida albicans in murine peritonitis. Infect Immun Vol. 67. p. 6637-42.
18. Girish Kumar C. P., S. S. Jothi Kumar and T. Menon. 2006. Phospholipase and proteinase activities of clinical isolates of Candida from immunocompromised patients. Mycopathologia Vol. 161. p. 213-218.

1. A herbal pharmaceutical composition comprising a biologically effective ethyl acetate extract of Lawsonia inermis, wherein the ethyl acetate extract comprises:
a total Phenolic content in an amount ranging from 0.00001 to 30%;
a total Flavonoid content ranging from 0.00001 to 40%; and
at least one compound selected from the group consisting of Lawsone; lawsoniaside and luteoline in an amount ranging from 0.00001 to 99%,
together with one or more pharmaceutical excipients, for the management of a fungal infection.

2. The herbal pharmaceutical composition according to claim 1, wherein the ethyl acetate extract comprises 0.01% to 99% of said at least one compound selected from the group consisting of Lawsone; lawsoniaside and luteoline.

3. The herbal pharmaceutical composition according to claim 1, wherein the ethyl acetate extract further comprises tannins.

4. The herbal pharmaceutical composition according to claim 1, wherein the fungal infection is selected from the group consisting of a systemic infection, a localized fungal infection, and a combination thereof.

5. The herbal pharmaceutical composition according to claim 1, wherein the composition further comprises one or more anti-fungal drugs selected from the group consisting of fluconazole, ketaconazole, clotrimazole, corbimazole, ravuconazole, posacconazole, Amphotericins, echinocandins, and mixtures thereof.

6. The herbal pharmaceutical composition according to claim 1, wherein the pharmaceutical excipients are selected from the group consisting of polymers, diluents, surfactants, binders, fillers, disintegrants, lubricants, colors, flavors, and combinations thereof.

7. The herbal pharmaceutical composition according to claim 1, wherein the extract is formulated in the form of a liquid, semisolid or solid dosage form.

8. The herbal pharmaceutical composition according to claim 7, wherein the extract is formulated for oral, topical, ocular, vaginal, otic, dermal application.

9. (canceled)

10. (canceled)

11. A pharmaceutical composition comprising lawsone, Lawsoniasides and Luteoline, either alone or in combination, in an amount of 0.0001% to 99.99% in association with one or more pharmaceutical excipients, for the management of fungal infections.

12. A method for treating candidiasis, comprising administering a therapeutically effective amount of an ethyl acetate extract of Lawsonia inermis in association with one or more pharmaceutical excipients.

13. The method according to claim 12, wherein the candidiasis is selected from the group consisting of oral candidiasis, vaginal candidiasis, and a combination thereof.

14. (canceled)

15. (canceled)

16. The herbal pharmaceutical composition according to claim 1, wherein the fungal infection is selected from the group consisting of oral candidiasis, vaginal candidiasis, and a combination thereof.

17. The herbal pharmaceutical composition according to claim 1, wherein the composition further comprises one or more anti-fungal drugs selected from the group consisting of Echinocandin, caspofungin, micafungin, anidulafungin and cilofungin.

18. A feminine hygiene product selected from the group consisting of pads and feminine wipes, comprising the herbal pharmaceutical composition according to claim 1.

19. A pharmaceutical formulation comprising the herbal pharmaceutical composition according to claim 1, wherein the formulation is a suppository; a toothpaste; a mouth wash; or a vaginal contraceptives or lubricant.

20. A process for the preparation of a biologically effective ethyl acetate extract of Lawsonia inermis, comprising;
a) shade drying green leaves of Lawsonia inermis and crushing the dried leaves into a fine powder;
b) soaking the crushed powder of green leaves of Lawsonia inermis with ethyl acetate in a ratio of between 10:1 and 1:10 overnight, followed by mixing for 3 hours using a stirrer with rpm of 100-150 to produce an extract solution;
c) filtering the extract solution to separate plant debris from a filtrate; and
d) removing the solvent from the filtrate by heating at 60° C. to produce a dark green colored viscous solution which has a desired concentration of the biologically effective ethyl acetate extract.
